(19) United States
(12) Patent Application Publication
(10) Pub. No.: US 2023/0263646 A1
(43) Pub. Date: Aug. 24, 2023

(54) SUPPORT STRUCTURE

(71) Applicant: SGL CARBON SE, Wiesbaden (DE)

(72) Inventors: Denis HINZ, Meitingen (DE); Fabian SCHUBERT, Meitingen (DE); Jochen SCHUTZBACH, Meitingen (DE)

(73) Assignee: SGL CARBON SE, Wiesbaden (DE)

(21) Appl. No.: 18/009,162

(22) PCT Filed: Jun. 8, 2021
(86) PCT No.: PCT/EP2021/065298

§ 371 Date: Dec. 8, 2022

(30) Foreign Application Priority Data
Jun. 9, 2020 (DE) 10 2020 207 216.7

(51) Int. Cl. A61F 2/78; A61F 2/50; A61F 2/80

(52) U.S. Cl. CPC A61F 2/7812; A61F 2/80; A61F 2/5046; A61F 2002/5052; A61F 2002/5053; A61F 2002/5056

(57) ABSTRACT

A support structure, a prosthesis component, and a method for producing a prosthesis component.

### FIELD

The present invention relates to a support structure, to a prosthesis component containing said support structure, to a method for producing this prosthesis component, and to the use of the support structure.

### BACKGROUND

In medicine, an artificial replacement of limbs or body parts is referred to as a prosthesis. The aim is for the prostheses to be as similar as possible in function to the organ or body part. Generally known prosthesis variants are exoprostheses (outside the body) and endoprostheses (completely enclosed by body tissue). Exoprostheses are, for example, leg, foot, arm or hand prostheses. A leg prosthesis, for example, has three main components, namely a shaft via which the prosthesis is connected to a residual extremity stump, an adapter piece, and a foot part. In addition to prostheses, so-called orthotics are also known. These serve to stabilise, relieve, immobilise, guide or correct limbs or the torso. Further developments of orthotics are so-called exoskeletons, which are robots or machines that can be worn on the body and which support or amplify the movement of the wearer.

Creating trusses as a support structure is a well-known approach in the art to stabilise structures. In medical technology for human and veterinary medicine, support structures are used to stabilise and reduce fractures, or to build up bones. The support structures can be made of titanium, a plastics material or steel, as well as resorbable materials. The disadvantage in this case, however, is that although individual reinforcement is technically possible, it is associated with high costs and complex process steps.

### SUMMARY

The object of the present invention is therefore to provide a support structure which, for example when used in a prosthesis, offers targeted reinforcement, so that material is saved and the wearing comfort for the wearer of the prosthesis is increased by the targeted reinforcement - and also a targeted rigidity and strength are achieved.

According to the invention, a support structure is provided which comprises at least one composite material, the support structure being designed as a shell-shaped body, 3D structure, or free-form surface, preferably as a shell-shaped body.

The support structure described is not a structure covering a complete surface - but rather a support structure that is only present in specific, required locations and therefore only covers a partial surface.

Within the scope of the invention, a fibre-reinforced plastics material is considered as a composite material, the fibres being selected from the group of carbon fibres, glass fibres, aramid fibres, metal fibres, ceramic fibres, basalt fibres, or natural fibres, preferably carbon fibres. Natural fibres are understood to mean flax, jute, sisal and hemp fibres. The fibres can be used as short, long, continuous fibres or mixtures thereof, preferably continuous fibres. Fibres with a length of 0.1 mm - 1 mm are referred to as short fibres; those with a length of 1 mm - 50 mm are referred to as long fibres, and those with a length of more than 50 mm as endless fibres. For the fibre-reinforced plastics materials, duromers such as epoxy resins, thermoplastics such as polyamides, and elastomers such as acrylonitrile-butadiene rubber or mixtures thereof, preferably duromers, are used as the matrix. Thermoplastics are plastics materials that can be deformed within a certain temperature range; the process is reversible as long as the thermoplastic does not decompose due to thermal overheating. In a preferred embodiment, the support structure comprises a carbon fibre-reinforced plastics material, the carbon fibres being in the form of endless fibres and the matrix being in the form of a duromer. This reinforcement material offers the advantage of low weight, very high weight-specific stiffness and tensile strength, as well as low thermal expansion which can be ideally adapted to the load by an individualised configuration of the fibre direction, and therefore distinguishes itself with excellent, adjustable mechanical properties for the named application.

A 3D structure is understood to mean any three-dimensional structure. A shell-shaped body is a 3D structure; it is designed to be open on one side and has at least one curved surface, so that a halved hollow sphere is formed. Free-form surfaces are understood to mean three-dimensional, curved surfaces. Curved surfaces are surfaces that deviate from a straight surface by any number of degrees in subregions of the surface.

According to a further advantageous embodiment, the composite material is selected from the group of carbon fibre-reinforced plastics material, glass fibre-reinforced plastics material, aramid fibre-reinforced plastics material, metal fibre-reinforced plastics material, ceramic-fibre reinforced plastics material, basalt fibre-reinforced plastics material, natural fibre-reinforced plastics material or mixtures of these, preferably carbon fibre-reinforced plastics material. The composite material made of carbon fibre-reinforced plastics material has the highest weight-specific stiffness.

A prosthesis component is understood to mean one or more components that are used in prostheses of any kind. The support structure allows for targeted reinforcement in the regions where it is needed. This increases the wearing comfort of the prosthesis for the wearer.

A further object of the invention is a prosthesis component comprising a sheath and the support structure, the support structure being attached to the sheath on the inside and/or outside, preferably on the outside. Within the scope of the invention, the sheath is understood to mean a shell-shaped body, a 3D structure or free-form surface, preferably a shell-shaped body, the shape of the sheath being dependent on the shape of the support structure. In a prosthesis component for a prosthetic leg, the sheath is a shell-shaped body, wherein the sheath surrounds the amputation stump. The various support structures allow for targeted reinforcement, adapted to each prosthesis component and to the wearer of the prosthesis, increasing the wearing comfort and creating a prosthesis that lasts longer.

According to the invention, the sheath is made of a plastics material, ceramic, metal or mixtures of these, preferably a plastics material. Plastics materials have the advantage that the sheath is particularly flexible, which increases the wearing comfort of the prosthesis for the wearer. Furthermore, the use of a plastics material allows a corresponding manufacturing process to be used in a comparatively simple, quick and cost-effective manner.

The support structure advantageously has at least two mouldings. In the context of this invention, mouldings are understood to mean subregions that are separated by regions that have no support structure. The mouldings have the advantage that only the regions that require support are supported, not by a full-surface support structure, but by a support structure applied in certain regions, which saves material costs and, in the case of a support structure that is used in prostheses, there is an improvement in the wearing comfort for the wearer, as this increases the flexibility of the prosthesis.

In a further advantageous embodiment, the mouldings of the support structure are connected to one another at a point, depending on the force load points. This means that the support structure does not have to be designed to cover the entire surface, but has support only at the required locations.

According to the invention, the sheath and the support structure are connected to one another in a non-positive and/or positive and/or material connection at this point, preferably in a non-positive and/or material connection. This is because, with a material connection, the forces are transmitted over the entire surface, and the non-positive connection can be released again - and it is possible to influence the connection strength.

Advantageously, in the case of a non-positive and/or positive and/or material connection of the sheath and support structure, a fastening means is selected from the group comprising screws or rivets for non-positive connections and/or from the group comprising adhesives for material connections, and/or clamps for positive connections, preferably screws and/or adhesives.

Adhesives are understood to be adhesive films with a thickness of 0 to 250 µm, adhesive foils with a thickness of >250 µm to 1 mm, or adhesive layers with a thickness of >1 mm to 5 mm, and adhesive pastes. Adhesive films, adhesive foils and adhesive layers are two-dimensional, flat structures with a homogeneous distribution of thickness. Adhesive pastes, for example, can be used to compensate for inaccuracies in the manufacture of the sheath and support structure. Fastening means are understood to mean both additional elements such as screws, rivets, adhesives, adhesive films, adhesive layers, adhesive foils or adhesive inserts, but also indirect fastenings such as by clamps, which is made possible by undercuts. Undercuts are understood to mean that the support structure extends over and around convex bulges on the sheath.

A further object of the invention is a method for producing the prosthesis component, comprising the following steps:
a) moulding the sheath by additive manufacturing, injection moulding or casting,
b) pretreating the surface of the sheath,
c) forming the support structure on the sheath surface pretreated in step b), wherein at least two layers each consisting of at least one composite material portion are applied,
d) curing or consolidating the support structure according to step c) at temperatures in a range from 80 to 150° C. and a pressure from 1 to 10 bar for 3 to 240 min,
e) separating the sheath and support structure,
f) post-processing the support structure,
g) bringing the sheath and support structure together,
h) connecting the sheath and support structure by means of a fastening means in a non-positive and/or positive and/or materially-bonded manner.

The sheath can be moulded in step a) using various shaping methods, such as additive manufacturing, injection moulding or casting. Within the scope of the invention, surface treatment of the sheath is understood to mean that the sheath is prepared in such a way that the support structure applied in step c) and cured in step d) can be separated from the sheath in step e). Release agents, waxes, films, or even separating layers can be used to pretreat the surface. Release agents are therefore low-viscosity, pasty substances or substances that can be applied as a spray, for example semi-permanent polymer resins, silicones or chlorofluorocarbons, which can be applied by wiping, spraying or dipping and form a thin film which serves as an adhesion barrier. Alternatively, waxes that are dispersed in solvents can also be used. In addition, it is also possible to provide the surface with films or thin separating layers, for example silicone or PTFE (polytetrafluoroethylene) films, which in the further step do not form a chemical/physical connection either with the sheath or with the reinforcement structure, and therefore can be removed again at any time.

In step c), the support structure is formed on the pretreated sheath surface. For this purpose, at least two layers each consisting of at least one composite material portion are applied. Within the scope of the invention, a composite material portion is understood to mean a composite material that has not yet hardened or has not yet been consolidated, which in the simplest case represents the overall shape of the support structure, and in this simplest case two layers thereof are applied one on top of the other. Layers are understood to mean a layer of a composite material made up of one or more composite material portions lying against one another. However, the support structure can also be assembled from many individual composite material portions in at least two superimposed layers. The not yet cured or not fully consolidated composite material is selected from the group of carbon fibre-reinforced plastics material, glass fibre-reinforced plastics material, aramid fibre-reinforced plastics material, metal fibre-reinforced plastics material, ceramic fibre-reinforced plastics material, basalt fibre-reinforced plastics material, natural fibre-reinforced plastics material or mixtures of these, preferably carbon fibre-reinforced plastics material.

Step c) can also be carried out by an automated method, for which purpose it is first calculated where the individual composite material portions are to be applied, and then these are applied accordingly with the aid of fibre placement methods.

In step d), the support structure is cured at temperatures in a range from 80 to 150° C. and a pressure of 1 - 10 bar for 3 to 240 minutes. The support structure thereby assumes its final shape and retains its strength.

In step f), the support structure is post-processed. This includes filing, milling, grinding and deburring. In addition, the support structure can also optionally be painted or sealed with a varnish, for example. This is for aesthetic purposes. The optional painting can also be carried out on the prosthesis component first.

Advantageously, the sheath in step a) is made of a plastics material, ceramic or metal, preferably a plastics material. Plastics material has the advantage that the sheath is very flexible and soft, which increases the wearing comfort of the prosthesis or orthosis for the wearer, for example in the case of prostheses or orthotics. Furthermore, the use of a plastics material allows comparatively simple, quick and inexpensive manufacture.

According to a further advantageous embodiment, the sheath in step a) is made of a plastics material selected from the group comprising thermoplastics, elastomers or duromers, preferably thermoplastics. Thermoplastics offer the advantage that they can be printed using 3D printing.

According to a further alternative method, the calculated thermal distortion during curing of the prosthesis component consisting of the sheath and the support structure is taken into account in step a) when the sheath is moulded and in step c) when the support structure is formed, so that steps b) and e) - g) are omitted. The thermal distortion can be calculated using various methods such as FEM models/simulation (finite element method), manual calculations, behaviour models, ROM (reduced order model), and is thus already included in the production of the sheath in such a way that it has a different shape from the measured model, and the desired geometry of the end component is achieved by means of the calculated distortion. The calculation of the thermal distortion also simplifies the manufacturing process of the entire component, as there is no need for time-consuming post-processing. In addition, no fastening means are required for the sheath and support structure, since the curing together already creates a material bond between the sheath and the support structure.

According to a further alternative method, in step a) the sheath is adapted on the inside according to the determined individual shape of the amputation stump, and the outer contour corresponds to a standard shape, and the support structure in step c) is formed on a separate tool, wherein at least two layers each consisting of at least one composite fibre material portion are applied, such that steps b) and e) are omitted. By using standardised outer contours, it is possible to produce support structures in advance, which increases cost-effectiveness; the individual adaptation is retained by adapting the inside of the sheath to the amputation stump.

Advantageously, in the case of a non-positive and/or positive and/or material connection of the sheath and support structure, a fastening means is selected from the group comprising screws or rivets for non-positive connections and/or from the group comprising adhesives for material connections, and/or clamps for positive connections, preferably screws and/or adhesives.

The support structure can generally be used in equipment for medical technology, rehabilitation aids, sports and veterinary medicine, as well as aids for static-dynamic loads, for patient recovery technology in the emergency field, and patient transport. In particular, the support structure is used to reinforce prostheses, orthotics, exoskeletons, mobility aids, surgical equipment, helmets and neck supports. In all of these uses, the support structure comprises the composite materials described above.

### BRIEF DESCRIPTION OF THE FIGURES

In the following, purely by way of example, the present invention is described by way of advantageous embodiments and with reference to the accompanying drawings.

FIG. 1a is a perspective view of a sheath (1) and a support structure (3).

FIG. 1b is a perspective view of a prosthesis component comprising a sheath (1) and a support structure (3).

FIG. 2 is a cross section of a prosthesis component comprising a sheath (1) having standardised support structures (3), (3′) and (3″).

FIG. 3 is a perspective view of a sheath (1) and sheath (1′).

### DETAILED DESCRIPTION

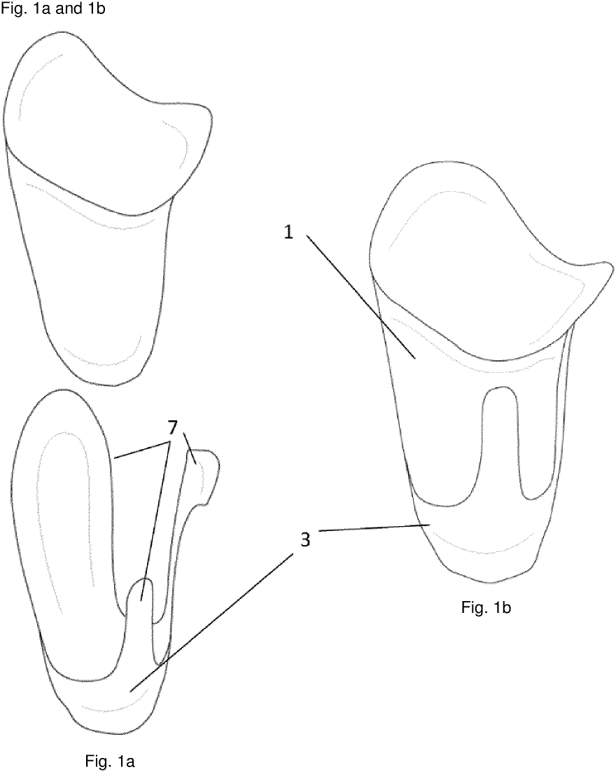

FIG. 1a shows a sheath (1) and a support structure (3) having mouldings (7).

FIG. 1b shows a prosthesis component comprising a sheath (1) and a support structure (3) having mouldings (7).

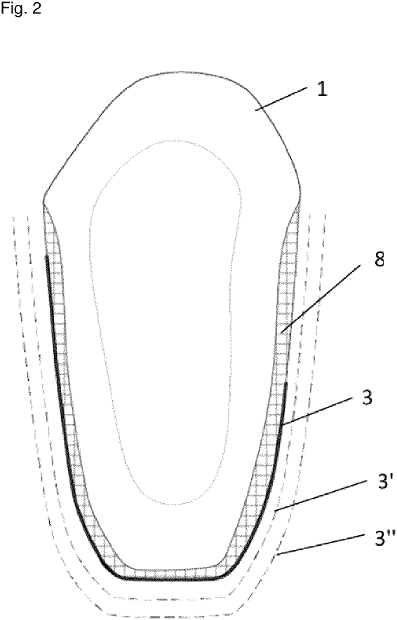

FIG. 2 shows a prosthesis component comprising a sheath (1) having standardised support structures (3), (3′) and (3″). Which of the standardised support structures (3), (3′) and (3″) is used for the prosthesis component depends on the size of the sheath (1), which is adapted to the amputation stump. In this case, it is the standardised support structure (3). The standardised support structures (3′) and (3″) are shown in this case only for the sake of clarity. The standardised support structure (3) is selected because the smallest thickening (8) is necessary compared to standardised support structures (3′) and (3″).

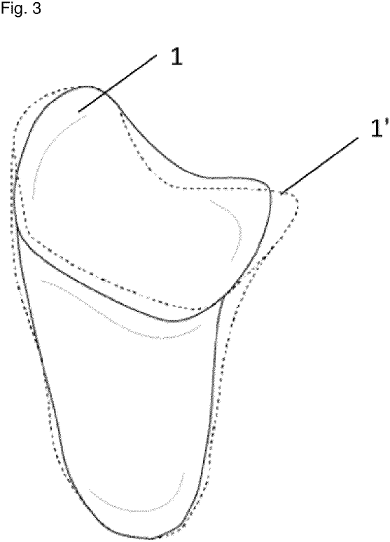

FIG. 3 is a perspective view of a sheath (1) and a sheath (1′). The sheath (1) represents the final sheath. The sheath (1′) contains, for example, the thermal distortion calculated for the sheath (1), so that the sheath (1) is obtained after the hardening of the support structure (not shown) with the sheath (1′).

The present invention is explained below using embodiments which, however, do not represent any limitation of the invention.

A prosthesis component can be produced as described below.

### Embodiment 1

The patient’s amputation stump was scanned and processed digitally, with the appropriate software being used to generate the geometry of the sheath (1) to be printed for the prosthesis component, and also the shape and thickness of the required support structure. Based on this model, a personalised sheath (1) made of a plastics material (polyamide 12) was produced using a 3D printing process. In the further process, this sheath (1) also served as a tool for the production of the support structure (3). For this purpose, the sheath (1) was covered with a 0.1 mm thick PVA (polyvinyl alcohol) film (separating layer) (2) without any creases. To produce the support structure (3), the support structure was laid in layers at the calculated points using an automated placement method, by alternating the arrangement of four composite material portions (4) (uncured composite material on a duromer epoxy resin basis with endless carbon fibres with a length of 55 mm) in 0/90° and +45/-45° orientation. In the next step, composite material portions were cured to form the support structure. For this purpose, the sheath (1) with the separating layer (2) applied and the composite material portions (4) laid down to form the support structure were cured in an autoclave process with a heating ramp of 2 K/min at 110° C., at a pressure of 6 bar, for a holding period of two hours. The resulting support structure (3a) was then separated from the sheath (1) by using the separating layer (2). Based on the data from the digital model, the edge contour and the surface of the support structure (3a) were then processed using a milling procedure, resulting in the support structure (3b). This was sealed with a varnish before the support structure (3) was connected to the sheath (1) in the subsequent step. For this purpose, a thixotropic 2-component PUR adhesive (5) was applied to the support structure (3) as a fastening means, the support structure (3) was connected to the sheath (1) and cured under mechanical contact pressure for 24 hours, so that a materially-bonded prosthesis component was obtained.

### Embodiment 2

The patient’s amputation stump was scanned and processed digitally using software to determine the geometry of the required sheath (1) as an individual prosthesis component, and the shape and thickness of a required support structure (3). In order to manufacture the support structure (3) directly on the sheath (1), the expected thermal distortion was determined using an FEM model and included in the design of the sheath and support structure in such a way that the components could be produced in a way that deviates from the shape determined by scanning; and the distortion was used to bring the combination of support structure and sheath into the final desired shape. The model of the sheath (1′) determined in this way was produced from a plastics material (polyamide 12) using a 3D printing process. To produce the support structure (3), the support structure was laid in layers on the sheath (1′) at the calculated points using an automated placement method, by alternating the arrangement of four composite material portions (4) (uncured composite material on a duromer epoxy resin basis with endless carbon fibres with a length of 55 mm) in 0/90° and +45/-45° orientation. In the next step, composite material portions were cured to form the support structure (3). For this purpose, the sheath (1′) with the pre-cut parts (4) laid to form the support structure was cured in an autoclave process with a heating ramp of 2 K/min at 110° C., at a pressure of 6 bar, for a holding period of two hours. Due to the direct contact of the two materials, the sheath (1′) and the pre-cut parts (4) laid to form the support structure, the material is distorted to produced the desired final geometry simultaneously with the adhesion between the support structure and the shell due to the adhesive properties and the penetration of the resin into the shell material during curing. For aesthetic reasons, the prosthesis component was painted with a varnish. An individually adapted and materially-bonded prosthesis component consisting of a sheath (1) and a support structure (3) was thus produced.

### Embodiment 3

The patient’s amputation stump was scanned and processed digitally, with the appropriate software being used to generate the geometry of the sheath (1) to be printed for the prosthesis component, and also the shape and thickness of the required support structure (3). The inner contour of this sheath (1) had a precisely fitting surface for the scanned amputation stump, but was adapted on the outside by a thickening (8) so that this outside corresponds to a previously defined standard shape. Based on this model, a sheath (1) was made from a plastics material (polyamide 12) using a 3D printing process. A plurality of models of the standard shape of the outer contour mentioned were defined independently of the scan mentioned. This standard shape could be used as a tool (6) on which the support structure (3) required for the sheath (1) was manufactured. The tool (6) was prepared in advance using a release agent (semi-permanent polymer resin) in order to ensure subsequent detachment of the support structure (3) from the tool (6). To produce the support structure (3), the support structure was laid in layers on the prepared tool in a targeted manner using an automated manufacturing process, by alternating the arrangement of four composite material portions (4) (uncured composite material on a duromer epoxy resin basis with endless carbon fibres with a length of 55 mm) in 0/90° and +45/-45° orientation. In the next step, composite material portions were cured to form the support structure (3). For this purpose, the tool (6) with the composite material portions (4) laid to form the support structure was cured in an autoclave process with a heating ramp of 2 K/min at 110° C., at a pressure of 6 bar, for a holding period of two hours. The resulting support structure (3a) was then separated from the tool (6) provided with a release agent. Then, based on the data from the digital model, the edge contour and the surface of the support structure (3a) were processed using a milling process, resulting in the final support structure (3). A thixotropic 2-component PUR adhesive (5) was applied to the support structure (3) as a fastening means, the support structure (3) was connected to the sheath (1) and cured under mechanical contact pressure for 24 hours, so that a prosthesis component was obtained.

List of reference signs

| | |
|---|---|
| 1, 1′ | sheath |
| 2 | separating layer |
| 3, 3′, 3″ | support structure |
| 3a | support structure before processing |
| 3b | support structure after processing |
| 4 | composite material portions |
| 5 | adhesive |
| 6 | tool |
| 7 | mouldings |
| 8 | thickening |

1-15. (canceled)

16. A support structure, comprising: at least one composite material, wherein the support structure is a shell-shaped body, 3D structure or free-form surface.

17. The support structure according to claim 16, wherein the composite material is selected from the group comprising carbon fibre-reinforced plastics material, glass fibre-reinforced plastics material, aramid fibre-reinforced plastics material, metal fibre-reinforced plastics material, ceramic fibre-reinforced plastics material, natural fibre-reinforced plastics material or mixtures of these.

18. A prosthesis component, comprising: a sheath and the support structure according to claim 16, wherein the support structure is attached to the shell on the inside and/or outside.

19. The prosthesis component according to claim 18, wherein the sheath is made of a plastics material, ceramic, metal or mixtures of these.

20. The prosthesis component according to claim 18, wherein the support structure has at least two mouldings.

21. The prosthesis component according to claim 20, wherein the mouldings of the support structure are connected to one another at a point, depending on the force load points.

22. The prosthesis component according to claim 21, wherein the sheath and support structure are non-positively and/or positively and/or materially connected to one another at this point.

23. The prosthesis component according to claim 22, wherein, in the case of a non-positive and/or positive and/or material connection of the sheath and support structure, a fastener is selected from the group comprising screws or rivets for non-positive connections, and/or from the group comprising adhesives for material connections, and/or clamps for positive connections.

24. A method for producing a prosthesis component according to claim 18, comprising the following steps:
a) moulding the sheath by additive manufacturing, injection moulding or casting,
b) pretreating the surface of the sheath,
c) forming the support structure on the sheath surface pretreated in step b), wherein at least two layers each consisting of at least one composite material portion are applied,
d) curing the support structure according to step c) at temperatures in a range from 80 to 150° C. and a pressure of 1 to 10 bar for 3 to 240 min,
e) separating the sheath and support structure,
f) post-processing the support structure,
g) bringing the sheath and support structure together,
h) connecting the sheath and support structure by a fastener in a non-positive and/or positive and/or materially-bonded manner.

25. The method according to claim 24, wherein the sheath in step a) is formed from a plastics material, ceramic or metal.

26. The method according to claim 25, wherein the sheath in step a) is made of a plastics material from the group comprising thermoplastics, elastomers or duromers.

27. The method according to claim 24, wherein in step a), when the sheath is moulded, and in step c) when the support structure is formed, the calculated thermal distortion during curing of the prosthesis component made of the sheath and support structure is taken into account, so that steps b) and e) - g) are omitted.

28. The method according to claim 24, wherein, in step a), the sheath is adapted on the inside according to the determined individual shape of the amputation stump, and the outer contour corresponds to a standard shape, and the support structure in step c) is formed on a separate tool, wherein at least two layers each consisting of at least one composite fibre material portion are applied, such that the steps b) and e) are omitted.

29. The method according to claim 24, wherein, in the case of a non-positive and/or positive and/or material connection of the sheath and support structure, the fastener is selected from the group comprising screws or rivets for non-positive connections, and/or from the group comprising adhesives for material connections, and/or clamps for positive connections.
